(19) United States
(12) Patent Application Publication
(10) Pub. No.: US 2003/0152612 A1
(43) Pub. Date: Aug. 14, 2003

(54) Method and article to control cellulite

(76) Inventors: Peter T. Pugliese, Pottsville Pike, PA (US); Kishore R. Shah, Bridgewater, PA (US)

Correspondence Address: Arthur R. Eglington, Esq., RD # 5, 113 Cross Creek Drive, Pottsville, PA, 17901, US

(21) Appl. No.: 10/329,944

(22) Filed: Dec. 27, 2002

Related U.S. Application Data
(60) Provisional application No. 60/344,276, filed on Jan. 3, 2002.

(51) Int. Cl. A61K 31/522; A61K 9/70; B32B 27/12

(52) U.S. Cl. 424/449; 514/263.34; 442/123

(57) ABSTRACT

A multilayer, flexible patch configured for the transdermal treatment of the skin of a human subject with an active ingredient, which ingredient is adapted for reducing the incidence of human cellulite and other fatty tissues comprising a pressure sensitive, skin adherable first layer selected from one of the polymeric materials comprising acrylate copolymer, vinyl ether copolymer, and a silicone adhesive polymer. It contains, by dispersal therethrough, an active ingredient selected from the group consisting of xanthine, hypoxanthine, theophylline, 7-theophylline acetic acid, caffeine, and theobromine. There is an outer backing, second layer comprising a water-soluble, skin conformable fabric adapted to provide moisture vapor permeability so to permit transepidermal moisture escape therethrough. Lastly, there is provided a protective liner, third layer, which is substantially, non-adhesive as to the adjacent, normally skin-adherable first layer and is protective of the active ingredient.

### CROSS-REFERENCE TO OTHER APPLICATIONS

This is a continuation-in-part of our copending provisional Specification, filed Jan. 3, 2002, under U.S. Ser. No. 60/344,276.

### FIELD OF THE INVENTION

The present invention relates to a method and an article for the control and treatment of a cosmetic condition known as cellulite. The invention specifically relates to a transdermal patch and its topical application to skin for the reduction of cellulite and other fatty tissues at the site of application.

### BACKGROUND OF THE INVENTION

Cellulite is a term imported from the French to describe a condition of the buttocks and upper thighs characterized by an unattractive, undulating, irregular skin surface. The condition has been thought by previous investigators to originate from abnormal fatty deposits that are collected under the skin. Many attempts have been made to define cellulite but no adequate explanation has been forthcoming. While fat is not the major etiological factor in cellulite, it is an important component of the condition.

The mechanism underlying cellulite appears to be as follows:

1. At the time of puberty the estrogen level rises, causing a stimulation of fibroblasts to produce collagenase.

2. Collagenase specifically attacks the superficial fascia and other collagen tissues, producing a weakening of the collagen fibers and allowing an area of broken fascia to appear. Fatty tissue herniates through this fascia and pushes upwards into the dermis.

3. The dermis is also weakened by the action of collagenase on the collagen fibers in the dermis, and thus offers no resistance to the upward movement of the fatty tissue. The net result is the undulating appearance of the outer skin as the fat pushes upwards.

4. The fatty tissue produces more estrogen, resulting in greater fibroblast activity and more collagenase being produced. As a result, cellulite is a progressive, self-perpetuating condition.

Thus, cellulite is an accumulation of fatty tissue irregularly distributed in thighs and buttocks of women. It is well documented that these female fat cell (adipocytes) are special fat cells that are actually fat deposits reserved for pregnancy and lactation. This fatty tissue is under special hormonal control and is not used for general energy supply, as is the fat on the abdomen and elsewhere. Therefore, exercise and other metabolic activities alone cannot reduce cellulite. Topical treatment with one or more physiologically active compounds is directed at controlling the breakdown of collagen and reducing the fat mass to a smaller volume.

It is known that a class of such organic compounds, known as xanthines, can reduce fatty tissue in the underlying skin if applied topically. The most common xanthines are caffeine, theophylline, and theobromine. Theophylline and caffeine are the most common xanthines used in the treatment of cellulite. The action of a xanthine is to direct the body to utilize the fatty tissue wherever it is placed.

U.S. Pat. No. 4,288,433 to Koulbanis, et al, teaches using xanthine compounds to treat cellulite. U.S. Pat. No. 6,153,207 to Pugliese, has described an invention related to the use of a garment, such as a panty hose, to which theophylline, inter alia, is bound by means of a semi-durable chemical bonding sites created by covalent bonding of polyethylenimine on the surface of the fabric material. Theophylline molecules are released to the skin while the garment is worn. The release of theophylline is activated by normal skin conditions such as pH, moisture, and body heat. Theophylline-releasing panty hose for the treatment of cellulite is commercially available under the trademark Cellulite® hosiery. Another xanthine (caffeine) product for this use is also available in the form of a cream (ROC™ Anti-Cellulite cream, Johnson & Johnson).

While these methods, if appropriately practiced, can address the cosmetic need of women to reduce cellulite, there are certain limitations in each method. For effective reduction of cellulite, a xanthine has to be delivered at the site for an extended period of time, and under appropriate metabolic and dietary conditions. Even then, it takes several weeks to months for the attainment of measurable and observable reduction of cellulite. A critical deficiency of the cream formulation is that it is not effectively retained at the site. It is easily rubbed off by articles of clothing. Therefore, continuous topical delivery of theophylline by a cream for a long duration of time is not practical. Likewise, the use of panty hose around the clock for weeks and months is neither convenient nor economical.

It is a principal object of this invention to provide a novel device/article, and a method of treatment, that can address the deficiencies of those teachings of the prior art. Another object of the present invention is to provide a device in the form of a transdermal patch, which can topically deliver a selected xanthine to skin in a continuous manner, for one or more days, and is at once convenient to use and does not adversely affect normal skin functions, including transepidermal water loss.

### SUMMARY OF THE INVENTION

According to the present invention, a treatment protocol for a cosmetic condition in females, known as cellulite, involves novel utilization of transdermal delivery of an effective agent, like xanthine onto the female outer skin for reducing cellulite. The means to effect such transdermal deposit is a flexible patch of variable dimensions and of essentially two layers, the one layer of which contains one or more active agents layer, and with a backing layer such as are used in adhesive tape for applying dressings to patient lesions. The active layer is a pressure-sensitive adhesive layer which contain a biological agent. Lastly, there is provided an inner surface placed, releasable liner component. The liner is adhered during patch fabrication, for active agent preservation, but which such liner is removed just prior to patch application to the affected human skin.

The present invention is directed to an article, in the form of transdermal patch, and a method using such for the reduction of cellulite and other fatty tissues at the topical site of its application on a human skin. The transdermal patch is comprised of a multilayered construction. The patch must have at least two layers of sheet materials, a first layer and a second layer, tightly adhering to one another (FIG. 1). The first layer comprises a pressure-sensitive, adhesive layer containing an adequate amount of a selected xanthine, uniformly distributed therein, for delivery to the skin over the duration of the patch application. The second layer consists of a backing film or a fabric, which provides protection to the xanthine containing adhesive first layer when applied to skin. Generally, the patch construction also has a third layer in the form of a releasable liner (FIG. 2), which provides protection to the first layer during storage and facilitates handling. The third layer is removed just prior to the patch application to skin. The patch can be applied to the desired skin site by means of the adhesive first layer, which tightly adheres to the skin. Then, xanthine from the patch continuously diffuses through the skin into the underlying tissues including the fatty tissues.

### DESCRIPTION OF FIGURES

FIG. 1 depicts the two layer patch precursor of the present invention;

FIG. 2 depicts a first embodiment of the multilayer patch of the invention; and

FIG. 3 depicts an alternate embodiment of the multilayer patch of the present invention.

### DETAILED DESCRIPTION OF THE PREFERRED EMBODIMENT

The xanthine-dispensing patch of this invention may have any dimensions that are convenient to the user. The xanthine patch may be applied to skin continuously for a period of time as long as seven days. It is well known that when the skin is occluded by an article, such as a Saran film or a patch, such a section of skin gets macerated because the normal transepidermal water loss is either prevented or restricted. This maceration is especially severe and damaging to the skin when the occlusion of the skin occurs for many days. The xanthine-dispensing patch of this invention has a moisture vapor permeability of at least 300 grams of water/sq. meter/24 hours at 37° C. and 100% relative humidity conditions (ASTM E-96 or related test method). This moisture vapor permeability of the patch allows normal transepidermal water loss to occur, and thereby prevents skin maceration, even when a large xanthine-dispensing patch is allowed to stay in tight contact with the skin for as long as seven days.

Xanthine itself has a chemical structure known as 2,6-dihydroxypurine, which has as number of known analogs and derivatives. Some common xanthines, which may be usefully employed for use in this invention are caffeine (1,3,7-trimethylxanthine), theophylline (1,3-dimethylxanthine), 7-theophylline acetic acid, and theobromine (3,7-dimethylxanthine). Theophylline is a particularly preferred xanthine for use in the transdermal patch of this invention.

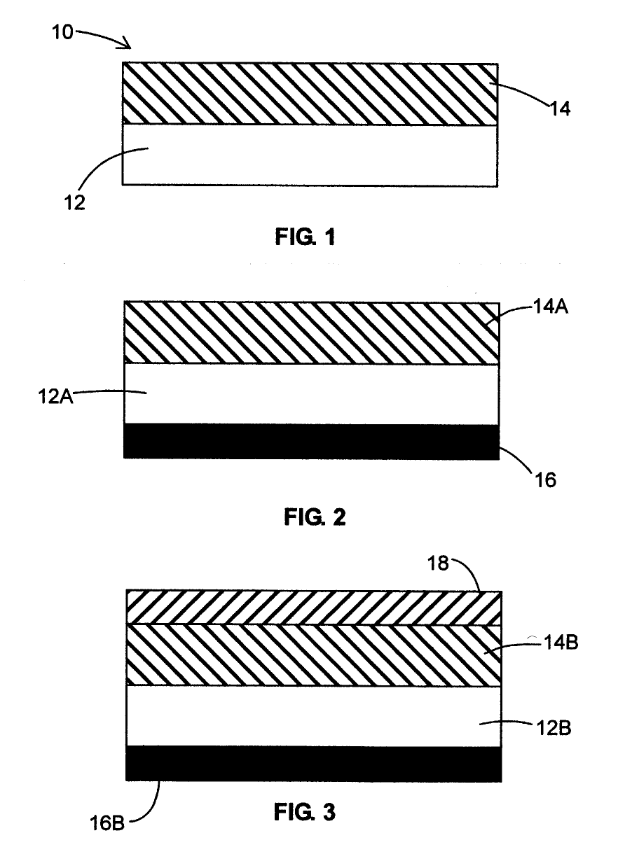

Referring now to FIG. 1, the useful dual layer patch of the invention, generally 10, is depicted. The initially inner first layer 12 is seen, which layer is the pressure sensitive adhesive layer for the skin. The overlying outer layer 14 is the backing layer which functions as described herein. It remains laminated to the first layer 12 during the skin adhesion period.

In FIG. 2, the third inner layer 16 has been added, serving as a manually releasable protective liner. Outer liners 12A and 14A are identical to like layers in the patch of FIG. 1.

An alternate embodiment is depicted in FIG. 3, presenting an optional outermost, flexible layer 18, which functions as a supporting layer, lightly adhering to adjacent layer 12B. This feature facilitates separation of the more adherable underlying layers.

The xanthine-containing, adhesive first layer of the patch may be selected from an acrylate copolymer, a vinyl ether polymer, or a silicone adhesive polymer. These pressure-sensitive adhesives are readily available from commercial suppliers such as Solutia, National Startch & Chemicals, BASF Corporation, and Dow Corning Corporation. Although the said polymers are preferred for use in this invention, other pressure-sensitive adhesive compositions may alternately be used. The thickness of the first layer may range from about 1 mil to 2 mils, and preferably from about 1 mil to about 1.4 mils. A selected xanthine is uniformly dispersed and/or dissolved in the adhesive layer. The patch typically contains a concentration of the xanthine in amounts represented by 10-600 micrograms/cm², depending upon duration of its intended application to skin. For a seven day use, the preferred concentration of the xanthine in the patch may range from 50 to 400 micrograms/cm², and preferably from 100-250 micrograms/cm². The patch may contain lower concentrations of the xanthine for shorter periods of use.

The second layer (backing layer) for the xanthine delivery patch may be of a water-resistant, skin-conformable fabric, or of an elastic film, either of which will also provide adequate moisture vapor permeability, so as to allow normal body flexing, and to permit transepidermal water loss. A type of moisture vapor permeable elastic film suitable for use in this invention is a thermoplastic polyether polyurethane film, which is commercially available from a variety of suppliers, such as Mylan Laboratories and Norwood Coated Products (a division of Saint-Gobain Performance Plastics). Thickness of the useful common polyurethane films should be about 1 mil. Moisture vapor permeability of the film nay range from about 500 to 2000 grams, and preferably from about 700 to more than 2000 grams, of water/sq. meter/24 hours, at 37° C. and 100% relative humidity conditions. Non-woven, woven, or knitted fabrics, are also generally useful as a backing layer of the present patch, because their macroscopic porosity permits the transdermal epidermal water loss. Thin polyurethane film, described herein, is generally preferred for use in this invention as the backing layer.

The third layer (a protective release liner) is typically of a reconstituted cellulose material, like bond paper, or may be of a more durable cast polyester film, which film is then precoated as a contiguous film with a silicone polymer. Such polymer does not exhibit adhesion to the acrylic and vinyl ether pressure-sensitive adhesive first layer containing the disposed xanthine. When a silicone coated, pressure-sensitive adhesive is used, the release coating is generally comprised of a fluorocarbon polymer. Use of the release liner provides for convenience of patch handling, and protects the intermediate layer-disbursed xanthine from topical soiling, or other contaminants infiltration, during the product transport and storage phase. The release liner (third layer) is easily removed by manual peeling at patch application time, and is discarded.

When an elastic polyurethane film is used as the backing layer, an optional supporting layer (fourth layer), comprised of a release liner or a plastic film, lightly adhering to the second layer (FIG. 3), on the side opposite to that of the first layer, may be employed in construction of the xanthine patch. When the release liner from the adhesive side is removed, the elastic patch is subject to sticking to itself, if it is not properly manipulated. The supporting layer facilitates handling of the sticky elastic patch in the process of its application to skin. After the patch is securely applied to the skin, the supporting layer is easily peeled away.

The transdermal patch of this invention is prepared by casting a homogenous dispersion or solution of the selective xanthine in a solution of the pressure-sensitive adhesive material in a volatile organic solvent; e.g., ethyl acetate, methyl ethyl ketone, toluene, etc., onto the release liner component, drying the same to remove solvent, and then laminating one cast surface of the xanthine-containing adhesive layer to the backing layer, either a fabric or a film. The release liner is, of course, adhered to the opposing surface of the adhesive layer.

The effectiveness of a xanthine-containing transdermal patch, or any other method of treatment in reduction of sub-dermal cellulite and other fatty tissues at the skin site of application, is related to the ability of the method to provide the required amounts of the xanthine to the tissues by a process of diffusion through the skin. It is an objective of the present invention to provide for transdermal permeation by the xanthine continuously, for a period of time ranging from one day to seven days, at a rate of about 2 to 50 micrograms/cm²/day. It is well known that the rate of permeation of xanthine-molecules through skin may vary significantly from subject to subject. Therefore, the above stated xanthine permeation rates are average values.

### WORKING EXAMPLES—IN VITRO and IN VIVO

### Examples 1-2

General Methods:

### Example 1

Xanthine Patch Preparation:

First, a paste containing finely dispersed containing a 50-60% xanthine (theophylline or theophylline 7-acetic acid) in poly(ethylene glycol) PEG 400) is prepared in a pestle/mortar. Then the required amount of the paste is uniformly dispersed in a known weight of the adhesive solution (Gelva 737, or a mixture of 75:25 Gelva 737/Gelva 788 adhesives) to yield a xanthine concentration in the range of about 2-8%, based on dry weight of the adhesive. Then the xanthine/adhesive mixture is cast on a release liner using a Gardener knife, equipped with two micrometer gap adjusting heads, at a gap setting of 10 mils. The adhesive coating is allowed to dry in the hood under an air current for a period of 30 minutes. The dried adhesive is then cured in an air-circulating oven at 85° C. for five minutes. MedifilM® 426 polyurethane film, having a nominal thickness of about 1 mil, is then manually laminated onto the adhesive coating, taking care not to entrap air bubbles between the adhesive and the polyurethane film. The laminated construction, thus prepared is kept in the air-circulating oven at 85° C. for an additional period of five minutes to optimize mass anchorage of the adhesive to the film. The prototypes thus prepared have xanthine-containing adhesive coating weight of about 3.65 mg/square centimeter (about 1.1-1.25 mil thickness) and the film/adhesive structure thickness of 2.1-2.25 mils. Depending on the concentration of the xanthine in the dry adhesive, the amount of xanthine in the patch may vary from 70-300 micrograms/cm².

### Example 2

In Vitro Skin Permeation Study of Xanthine

In vitro skin permeation studies of a xanthine-containing patch are done using epidermis of a dermatomed human cadaver skin (obtained from New York Fire Fighters Skin Bank, New York, N.Y., and stored in the freezer). Epidermis of the skin is carefully separated after the skin is first allowed to thaw, and then equilibrated in distilled water at 50-60° C. for˜1 minute. After the epidermis is dried by blotting with a tissue paper, a round section of a xanthine-containing patch is attached to it, the drug/adhesive matrix facing the epidermis. Excess epidermis surrounding the patch is trimmed with a scalpel. The patch/epidermis composite is then placed on the opening of the receptor compartment of a Franz diffusion cell (Crown Glass Company, Somerville, N.J.), with the epidermis side facing the receptor compartment. The receptor compartment is then filled with the receptor medium (10% aqueous acetonitrile solution) and continuously stirred by means of a magnetic stirrer. Temperature of the diffusion cell assembly, including the receptor fluid, was maintained at 32-34° C. (approximate skin surface temperature) by circulation of warm water through the jacket surrounding the cell using Haake circulating bath. The receptor fluid is changed at appropriate interval of time and analyzed for xanthine concentration by means of ultraviolet spectroscopy.

### Examples 3-7

Skin Permeation of Xanthine-Containing Transdermal Patches:

Theophylline and theophylline 7-acetic acid (TAA) patches were prepared as taught and their skin permeation from the patches were studied, by the methods described herein using epidermis of human donor cadaver skin (arbitrarily designated as X, Y, and Z). Theophylline skin permeation from each patch was plotted as cumulative amount permeated versus time up to seven days. A linear profile, indicating a constant skin permeation rate, was observed in all the experiments. The skin was found to be the controlling factor in transdermal permeation of theophylline and TAA from the inventive patch.

Xanthine flux from the patch was calculated as follows:

$$\begin{matrix}Flux , J = Slope \times 24 / Cell Area ( cell area = 0.69 {cm}^{2} ) \\ = micrograms of xanthine \text{/} {cm}^{2} \text{/} day \end{matrix}$$

The skin permeation rates (Flux, J) of the patches were found to be in the range of 6-40 micrograms/cm²/day (Table I).

### Example 8

Skin Permeation of Cellulite®) Pantyhose:

A control skin permeation experiment, using the Cellulite® pantyhose, having active ingredients are theophylline and silantriol per U.S. Pat. NO. 6,153,207, instead of the xanthine patch, was conducted in a similar manner for comparison purposes. Results of the experiment showed that theophylline flux, J from the anti-cellulite patch was about 2 micrograms/cm²/day.

Pugliese (U.S. Pat. No. 6,153,207) has disclosed and shown that theophylline releasing Cellulite® hosiery is effective in significantly reducing fatty tissues of cellulites. Therefore, a comparison of the results of the skin permeation studies of the xanthine patches and the Cellulite(t hosiery reported herein indicated that the theophylline patch is likely to be at least as effective as the Cellulite® hosiery.

### Example 9

Theophylline Patch/Human Patch Test:

The purpose of the human patch test was to determine the amount of theophylline present in the upper cellular layers of the stratum corneum, after application of a theophylline patch for a specified period of time. A theophylline patch having the following description was prepared by the general methods described herein:

| | |
|---|---|
| Adhesive: | Gelva 727/Gelva 788 |
| Theophylline Concentration | 252 micrograms/cm² |

In this study, the theophylline content of the upper cellular layers of the stratum corneum was determined by Scotch tape stripping of the skin, followed by assay of theophylline in the stripped skin. Seven (7) female subjects participated in this preliminary study. Five subjects had three 2×2 patches applied and adhered to dry skin of their upper outer thigh. Each patch was separated by ½-1 inches. The patches remained on the skin 48-72 hours. The other two subjects wore Cellulite® hosiery at least eight hours a day.

Scotch tape, brand #5930, was used to collect the tape strips of the stratum corneum. A strip of tape 2 cm by 8 cm was placed in the center of the test site and an even pressure was applied.

Cellulite® hosiery was distributed to two subjects. At approximately 8, 24, and 48 hours after wearing the hosiery, they reported back to the laboratory for the taking of tape strips. Adjacent areas on the thigh were used as the test sites.

Theophylline patches were applied on the thigh of the other five participants. At approximately 8, 24, and 48 hours after the application of the patches, subjects reported back to the laboratory. At each time point, the patches were removed from the skin in a uniform manner and a tape strip was collected.

To determine residual theophylline collected from the Scotch tape strips, the tape strips were cut to 2 cm by 5 cm and placed in separate jars that contained 3 mL of Dubelco's buffer. The jars were gently agitated every hour for 15 minutes. After three hours, the samples were analyzed for theophylline level on a Genesys II spectrophotometer at 274 nm.

Average values (five subjects) of the theophylline in the skin of subjects, wearing the patches at 8, 24, and 48 hour time points, were 40, 35, 28, and 30 micrograms/cm², respectively. However, no residual theophylline could be detected from tape strips collected from the two subjects that wore Cellulite® pantyhose.

These human in vivo results of transdermal permeation of theophylline from the theophylline patch are very consistent with the in vitro skin permeation studies reported herein. The results further indicate effectiveness, which is at least equal to that of the Cellulite® hosiery article, in reducing fatty tissues by topical application.

### Example 10

Moisture Vapor Permeability of a Theophylline Patch:

Moisture vapor permeability of a theophylline patch was determined by the desiccant cup method (modified ASTM-E96). Construction of the patch used was as follows:

| | |
|---|---|
| Backing: | 0.9 mil Medical Polyurethane Film |
| | (Norwood Coated Products) |
| Adhesive: | Gelva 737 |
| Theophylline Concentration: | 364 micrograms/cm² |

An amount of desiccant (granular anhydrous calcium chloride and molecular sieve absorbents) was placed in a cylindrical cup, so that the cup was ⅓ full. Then the mouth of the cup was covered with a sample of the theophylline patch, the adhesive side tightly sticking to the flange. The cup assembly was then weighed accurately.

The bottom surface of a chamber was filled with about 1″ layer of water. The chamber was sealed with its lid and placed in an oven maintained at 37° C. for 20 hours for equilibration. The cup assembly was then placed inside the chamber on a slightly raised platform.

The cup assembly is weighed 8, 24, and 48 hours after placing in the oven. The 48-hour weight measurement was taken mainly to ensure that the desiccant was not saturated within the first 24 hours.

Moisture vapor permeability was calculated as the gain in weight (water) per unit area in the first 24 hours at 37° C. under 100% relative humidity gradient. The moisture vapor transmission rate of the theophylline patch was found to be 1008 grams of water/meter²/24 hours (Table II).

### Example 11

Lipolyzer Fat Patch Study

The purpose of this study was to examine the theophylline patch of Example 10 for safety and efficacy and its ability to reduce the appearance of fatty skin tissue. The actual patch size is 4×6 inches.

Thirty female subjects participated in this study. Subjects had patches applied to abdominal or upper back thigh skin weekly. Twenty subjects had the patches applied weekly at our clinic and the other ten subjects applied and removed the patches at home daily. There were no exclusions in the subject's activity while the patches are in place. Subjects were instructed to wash, bath and dress as normal.

Subjects reported to the clinic weekly for assessments. Photographs utilizing the Mirror system were taken at baseline (day 1) and weekly up to eight weeks. We employed a Biosound AU5 ultrasound to obtain b-scans. Measurements (cm) of the abdomen or thighs were taken at the baseline visit and at four and eight weeks after treatment.

After one month, there was a 1.83 centimeter reduction in abdominal measurements.

After two months, there was a 0.11 centimeter additional reduction in abdominal measurements.

After one month, there was a 1.28 centimeter reduction in thigh measurements. After two months, there was a 0.31 centimeter added reduction in thigh measurements.

Photographs of the abdomen and thighs further confirm a reduction in fatty tissue. The ultrasound images are under evaluation.

Other modifications, variations, and improvements may become apparent to those skilled in the subject and related dermatological arts, once having studied the present specification, examples and claims.

TABLE I

| Ex- | | | | Xanthine | |
|---|---|---|---|---|---|
| ample | | Skin | | Conc. | Flux, J |
| No. | Xanthine | Donor | Adhesive | mcg/cm² | mcg/cm²/day |
| 3 | TAA | X | Gelva 737 | 275 | 9.8 |
| 4 | Theophylline | X | Gelva 737 | 267 | 10.2 |
| 5 | Theophylline | Y | Gelva 737/ | 267 | 39.9 |
| | | | Gelva 788 | | |
| 6 | TAA* | Y | Gelva 737 | 92 | 24 |
| 7 | Theophylline | Z | Gelva 737 | 259 | 7.8 |

*7-Theophylline acetic acid

TABLE II

| | | | | cup area | water vapor |
|---|---|---|---|---|---|
| | | | | (squarer | flux |
| | | | cup diameter | meters) | (g/square |
| Sample | g/h | g/24 h | (millimeters) | 0.1265 × 10⁻³ | meters/24 h) |
| 1 | 0.0053 | 0.1264 | 12.690 | 0.1265 × 10⁻³ | 999 |
| 2 | 0.0058 | 0.1335 | 12.7700 | 0.1265 × 10⁻³ | 1055 |
| 3 | 0.0051 | 0.1227 | 12.8800 | 0.1265 × 10⁻³ | 970 |
| Average | 0.0053 | 0.1275 | 12.7133 | 0.1265 × 10⁻³ | 1008 |

1. A multilayer, flexible patch configured for the transdermal delivery through the skin of a human subject of an active ingredient, which ingredient is adapted for reducing human cellulite and the reduction of other fatty tissues, comprising:
(a) a water vapor permeable, skin adherable, pressure-sensitive adhesive first layer containing by dispersal therethrough, a compound having a xanthine structure;
(b) an outer backing, second layer comprising a water vapor permeable, skin conformable fabric of a polymeric film adapted to provide moisture vapor permeability so to permit transepidermal moisture escape therethrough; and,
(c) a protective liner, third layer which is substantially non-adhesive as to the adjacent, normally skin-adherable first layer and is protective of the active ingredient.

2. The multilayer patch of claim 1 wherein the xanthine structure is selected from a group consisting of caffeine, theophylline, 7-theophylline acetic acid, and theobromine.

3. The multilayer patch of claim 1 wherein the pressure-sensitive adhesive is selected from a group consisting of an acrylate copolymer, a vinyl ether polymer, and a silicone adhesive polymer.

4. The multilayer patch of claim 1 wherein the second layer is adapted to lightly adhere to the exposed surface of the third layer, to facilitate storage and handling of the patch during application to the skin of the human subject.

5. The multilayer patch of claim 1 wherein the second layer is a thermoplastic polyether polyurethane film.

6. The multilayer patch of claim 1 wherein the third layer is selected from the group consisting of essentially reconstituted cellulose and cast polyester film pre-coated with a silicone polymer to preclude adhesion to the normally adhesive first layer.

7. The flexible patch of claim 1 wherein the first layer has a xanthine concentration range from 10-600 micrograms per square centimeter sufficient to provide for a transdermal permeation thereof into the skin for a period of time ranging up to seven days, at a permeation rate of 2-50 micrograms of xanthine per square centimeter of skin per day.

8. The flexible patch of claim 1 wherein the patch provides a moisture vapor permeability of at least 300 gm of water per square meter of said second layer in 24 hours, at 37° centigrade under 100% relative humidity gradient.

9. The flexible patch of claim 1 wherein the first layer has a transverse thickness ranging from 1-2 mils.

10. The flexible patch of claim 1 wherein the first layer has a xanthine concentration ranging from 10 to 600 micrograms per square centimeter of the first layer.

11. The flexible patch of claim 1 wherein the backing second layer is fabricated to provide a moisture vapor permeability ranging from about 500 to more than 2000 grams of water per square meter of said layer for 24 hours at 37° Centigrade and 100% relative humidity gradient.

12. A multilayer, flexible patch configured for effecting the transdermal delivery of the skin of a human subject with an active ingredient, which ingredient is adapted for reducing the incidence of human cellulite comprising:
(a) a pressure-sensitive, skin adherable first layer comprising an acrylate polymer substrate and theophylline dispersed therein as the active ingredient;
(b) an outer skin conformable, backing second layer comprising a thermoplastic polyether polyurethane copolymer film;
(c) a protective inner liner third layer which is substantially non-adhesive to the adjacent first layer comprising a silicone coated release paper or a durable cast polyester film; and
(d) a supporting fourth layer, which is a silicone coated release paper or a polymeric film lightly adhering to the second layer.

13. The multilayer patch of claim 1 wherein the releasable liner third layer is selected from the group consisting of reconstituted cellulose and cast polyester film, which is pre-coated with a silicone polymer adapted to inhibit adhesion of the third layer to the adjoining first layer, and to facilitate peeling for transdermal patch activation.

14. A method of treating skin with an active ingredient using a multilayer patch suited for adhesion to human skin of a subject and adapted for transdermal delivery of the active ingredient to the skin adhering for an extended periods, the patch consisting:
(a) A water vapor permeable, pressure-sensitive, skin adherable first layer containing a dispersed active ingredient, which is a compound having a xanthine structure;
(b) an outer backing, second layer comprising a water vapor permeable, skin conformable fabric of a polymeric film adapted to provide moisture vapor permeability of the patch so as to permit transdermal moisture loss to escape therethrough; and,
(c) a protective liner, third layer which is substantially, non-adhesive to the adjacent first layer and is protective of the active ingredient dispersed therein, and is adapted to be readily peeled from the patch at the time of skin application.

15. The multilayer patch of claim 14 wherein the xanthine structure is selected from a group consisting of caffeine, theophylline, 7-theophylline acetic acid, and theobromine.

16. The multilayer patch of claim 1 wherein a supporting fourth layer comprising a release liner adapted to lightly adhere to the outer surface of the second layer comprising a flexible polyurethane film.
